(19) United States
(12) Patent Application Publication
(10) Pub. No.: US 2024/0409477 A1
(43) Pub. Date: Dec. 12, 2024

(54) System and Method for E-Fuel Production

(71) Applicant: CATAGEN LIMITED, Belfast (GB)

(72) Inventors: Andrew Woods, Belfast (GB); Matthew Elliott, Carrickfergus (GB); Robert Douglas, Ballymena (GB)

(21) Appl. No.: 18/696,595

(22) PCT Filed: Sep. 29, 2022
(86) PCT No.: PCT/EP2022/077219

§ 371 Date: Mar. 28, 2024

(30) Foreign Application Priority Data
Sep. 29, 2021 (GB) 2113960.5

(51) Int. Cl. C07C 1/04; B01J 12/00; B01J 19/00; B01J 19/24; C01B 32/40; C10L 1/04

(52) U.S. Cl. CPC C07C 1/0485; B01J 12/00; B01J 19/0013; B01J 19/245; C01B 32/40; C10L 1/04; B01J 2219/00117; B01J 2219/00135; B01J 2219/002; B01J 2219/00243

(57) ABSTRACT

A system for producing synthetic fuel comprises a reactor having a first reaction zone for implementing a first reaction in which carbon dioxide and hydrogen react to produce carbon monoxide and water, and at least one other reaction zone for implementing a second reaction in which carbon monoxide and hydrogen react to produce a fuel precursor, and a third reaction involving synthesizing fuel from said fuel precursor. The reaction zones are inter-connected in series by a fluid circuit is configured to circulate and recirculate fluid around the reactor to facilitate recycling of reactants and heat energy. The system facilitates low-energy. cost-efficient production of liquid e-fuels.

### FIELD OF THE INVENTION

This invention relates to systems and methods for the production of synthetic fuels, especially electrofuels (e-fuels).

### BACKGROUND TO THE INVENTION

Electrofuels, or e-fuels, are synthetic fuels produced using renewable electrical energy. E-fuels are liquid or gaseous hydrocarbon fuels and may be produced from synthetic hydrogen (H₂) and carbon dioxide (CO₂) sequestered from the environment. Typically, the hydrogen is produced by using energy from renewable sources such as wind, wave or solar power, to break water down into its component parts, e.g. by electrolysis or a thermochemical cycle such as the sulphur-iodine cycle. Examples of e-fuels include e-hydrogen, e-methane, e-methanol, e-kerosene, e-petrol and e-diesel. E-fuels can be burned carbon-neutrally since no extra CO₂ is produced.

Aside from the environmental benefits, the drive for introduction of low carbon fuels is high due to global legislation requiring vehicles to reduce emissions and through governmental bodies providing funding for relevant solutions.

It would be desirable therefore to provide improved systems and methods for the production of synthetic fuels, especially e-fuels.

### SUMMARY OF THE INVENTION

From a first aspect the invention provides a system for producing synthetic fuel, the system comprising a reactor, the reactor comprising:
a fluid circuit;
means for driving fluid around said fluid circuit;
a first reaction zone for implementing a first reaction in which carbon dioxide and hydrogen react to produce carbon monoxide and water;
at least one other reaction zone for implementing a second reaction in which carbon monoxide and hydrogen react to produce a fuel precursor, and a third reaction involving synthesizing fuel from said fuel precursor;
means for introducing hydrogen into said fluid circuit;
means for introducing carbon dioxide into said fluid circuit; and
means for heating fluid in said fluid circuit,
   wherein said reaction zones are inter-connected in series by said fluid circuit, and wherein fluid circuit is configured to recirculate fluid around said fluid circuit.

In preferred embodiments, the reactor, including the fluid circuit, is configure to support circulation and/or recirculation of fluid around the reactor to facilitate recycling of unused reactants, and preferably also heat energy. As such, the preferred system facilitates low-energy, cost-efficient production of synthetic, or synthesized, i.e. manufactured, fuels, especially liquid e-fuels. In this context, a synthetic fuel may be any manufactured fuel as opposed to a natural fuel. It will be understood that embodiments of the invention may produce fuels that are not necessarily deemed to be e-fuels depending on how the energy and/or reactants used in the production process are sourced.

In preferred embodiments, said at least one other reaction zone may comprise a second reaction zone for implementing said second reaction, and a third reaction zone for implementing said third reaction, or may comprise a combined reaction zone for implementing both of said second and third reactions.

In preferred embodiments, the system comprises at least one reservoir for storing gas, said at least one reservoir being connected to said fluid circuit for delivery of gas into said fluid circuit and preferably also for receiving gas from said fluid circuit, wherein said fluid circuit is preferably configured to recirculate fluid from said at least one reservoir, through said reaction zones, and back to said at least one reservoir.

Said means for introducing hydrogen into said fluid circuit typically comprises means for introducing hydrogen into said fluid circuit at a location upstream of said first reaction zone and/or means for introducing hydrogen into said fluid circuit at a location between said first reaction zone and said at least one other reaction zone.

Said means for introducing carbon dioxide into said fluid circuit typically comprises means for introducing carbon dioxide into said fluid circuit at a location upstream of said first reaction zone.

In preferred embodiments, said reactor further includes at least one heat exchanger arranged to transfer heat from fluid exiting one or more of said reaction zones to fluid being delivered by said fluid circuit to at least one of said reaction zones, preferably at least said first reaction zone or only said first reaction zone. Said at least one heat exchanger is advantageously arranged to transfer heat from fluid exiting said first reaction zone and/or said at least one other reaction zone to fluid being delivered by said fluid circuit to said first reaction zone.

Said at least one heat exchanger may comprise a first heat exchanger arranged to transfer heat from said first reaction zone to fluid being delivered by said fluid circuit to said first reaction zone, and/or a second heat exchanger arranged to transfer heat from said third reaction zone or said combined reaction zone to fluid being delivered by said fluid circuit to said first reaction zone.

In preferred embodiments, said at least one heat exchanger comprises said first heat exchanger and said second heat exchanger, and wherein said fluid circuit is configured to deliver fluid to said first reaction zone via said first and second heat exchangers.

Preferably, said heating means comprises a first heating apparatus located upstream of said first reaction zone and being operable to heat fluid being delivered to said first reaction zone by said fluid circuit. Said heating means may comprise a second heating apparatus located between said first reaction zone and said at least one other reaction zone, and being operable heat fluid being delivered to said at least one other reaction zone by said fluid circuit.

The, or each, heating apparatus preferably comprises a furnace, preferably an electric furnace, more preferably a high thermal inertia electric furnace, or other electrically powered heating apparatus.

In preferred embodiments, the system includes means for controlling an amount of hydrogen and/or a ratio of carbon to hydrogen present in the respective reaction zone during implementation of said third reaction, preferably in order to determine the type of fuel synthesized from said fuel precursor.

In preferred embodiments, the system includes at least one, but typically a plurality of control zones are included in said fluid circuit at a respective different location, each control zone including at least one device for controlling at least one parameter of said fluid in accordance with control information and/or at least one parameter measurement device, the system further including a control system for controlling operation of the reactor, the control system being in communication with said control zones to provide each control zone with said control information and/or to receive parameter measurement information from the control zone. Said at least one parameter may comprises a respective parameter indicating any one or more of: fluid composition; fluid temperature; fluid flow rate; fluid pressure; fluid level.

Preferably, said control system is configured to calculate said control information by mathematically modelling said reactor using Model Predictive Control (MPC). Optionally, said control system is configured to determine said control information using a mathematical model of the reactor, and wherein said mathematical model preferably comprises a neural network model whereby said control system is configured to calculate said control information using an artificial neural network.

In preferred embodiments, said plurality of control zones includes a first control zone configured to control said at least one parameter of the fluid in said first reaction zone in order to implement said first reaction.

In some embodiments, said plurality of control zones includes a control zone configured to control said at least one parameter of the fluid in said combined reaction zone in order to implement said second reaction and said third reaction.

In some embodiments, said plurality of control zones includes a second control zone configured to control said at least one parameter of the fluid in said second reaction zone in order to implement said second reaction.

In some embodiments, said plurality of control zones includes a third control zone configured to control said at least one parameter of the fluid in said third reaction zone in order to implement said third reaction, or to control said at least one parameter of the fluid in said combined reaction zone in order to implement said second and third reactions.

Typically, the system includes means for separating the synthesized fuel from other fluid in said fluid circuit. Typically, the system includes means collecting and outputting the fuel from the reactor and/or for storing the fuel.

Typically, the system includes means for introducing a carrier gas into said fluid circuit.

From a second aspect the invention provides a method of producing synthetic fuel in a reactor comprising a fluid circuit, the method comprising:
introducing hydrogen into said fluid circuit;
introducing carbon dioxide into said fluid circuit;
implementing, in a first reaction zone of the reactor, a first reaction in which carbon dioxide and hydrogen react to produce carbon monoxide and water;
implementing, in at least one other reaction zone of the reactor, a second reaction in which carbon monoxide and hydrogen react to produce a fuel precursor, and a third reaction involving synthesizing fuel from said fuel precursor,
   wherein said reaction zones are connected in series by said fluid circuit and wherein said method further includes:
recirculating fluid around said fluid circuit.

Preferably, said implementing in at least one other reaction zone comprises: implementing, in a second reaction zone of the reactor, said second reaction; and implementing, in a third reaction zone of the reactor, said third reaction; or implementing, in a combined reaction zone of the reaction, said second reaction and said third reaction.

In preferred embodiments, the method includes controlling an amount of hydrogen and/or a ratio of carbon to hydrogen present in the respective reaction zone during implementation of said third reaction, preferably in order to determine the type of fuel synthesized from said fuel precursor.

In preferred embodiments, the method includes obtaining said hydrogen by implementing a thermochemical cycle in which water is split into hydrogen and oxygen. In preferred embodiments, the method includes sequestering said carbon dioxide from the atmosphere.

Preferred embodiments of the invention facilitate the commercial generation of synthesised fuel in a recirculating gas reactor by means of the Reverse Water-Gas Shift (RWGS) reaction and the Fischer-Tropsch (FT) processes (FIG. 1). Advantageously, using a recirculating reactor helps counteract any conversion inefficiencies in the RWGS reaction, where unconverted CO2 is not expelled, rather recirculated for conversion in the RWGS reaction zone during the 2^{nd}, 3^{rd} or Nth pass. Advantageously the system may operate at high temperature (>700° C.) which facilitates a high RWGS reaction rate and improved yield of RWGS products compared to lower temperature operation. Advantageously, the system supports a high degree of flow control, in particular the flow of carbon dioxide and hydrogen through catalysts at a high rate, minimising the power requirements of the overall process.

From one aspect, the invention provides a thermo-cyclic e-fuel production reactor. Advantageously, e-Fuel is produced using less energy and more cost efficiently than conventional solutions by utilising a multi-stage catalytic reaction process in a recirculating reactor. The preferred reactor is configured to implement the RWGS reaction and the FT process with the key reactants of CO₂ and H₂ being recirculated, thereby reducing waste products and increasing process efficiency. Preferably, the CO₂ is sequestered directly from the atmosphere, ensuring an even lower carbon, ideally zero carbon, solution for e-fuel production. Any method or system for sequestering CO₂ from the atmosphere, or from elsewhere in the environment may be used.

Advantageously, the recirculating gas reactor allows low-energy, cost-efficient production of a liquid e-fuel suitable for conventional combustion processes. In preferred embodiments, the production system includes a control systems that can create control zones within the reactor to facilitate each individual thermochemical reaction of the e-fuel production process.

Preferred embodiments of the invention provide an e-fuel producing system that is suitable for installation at renewal energy sites, e.g. wind farms. Producing e-fuels close to renewable energy sources is highly desirable, especially in conjunction with green hydrogen production, as the energy and H₂ can be transformed into e-fuel on site, which overcomes the problems associated with transporting gaseous H₂. Liquid e-Fuel is significantly more easily transported due to its high mass density and high specific energy density. For example, a fixed volume of e-Fuels typically has 7-8 times the energy content of the equivalent volume of hydrogen at a pressure of 350 bar.

In preferred embodiments, hydrogen and carbon dioxide are fed into a reactor forming a synthesis gas (CO and H₂) that is then synthesized to produce a fuel precursor, which is synthesized to produce an e-fuel, for example, e-diesel or e-kerosene.

E-Fuel production in in accordance with preferred embodiments releases relatively little carbon dioxide into the atmosphere when compared to fossil fuel or low-carbon alternatives. The preferred process is carbon neutral or net zero releasing only CO₂ that has previously been sequestered from the atmosphere. As the energy is totally renewable the fuel production can be considered as zero carbon.

Preferred embodiments of the system include a recirculating fluid reactor that is energy efficient and allows precise control of chemical composition, flow and temperature in one or more reaction zones where the reactants are converted to products by chemical reaction. Advantageously, mathematical model-based control is implemented at one or more control zones. Typically, operation of the reactor involves delivery of one or more gases and/or liquids into a closed system, or zone, of fixed known volume. Triangulation of multiple measurement sources, predictive models and calibrated gas/liquid delivery systems can ensure accuracy in a dynamic environment.

In preferred embodiments, the recirculating gas or liquid (fluid) production reactor comprises at least one, optionally two or more, recirculating gas systems/circuits with integral furnace(s), storage reservoir(s) and blower(s) or other fluid drive means. Heat is regenerated through an integral heat exchanger(s) and may be stored throughout the thermal inertia of the system.

Preferred embodiments of the invention provide precision controlled delivery of known quantity (ies) of gas(es) at a known time and location in the fluid circuit with a known concentration and a known temperature.

Advantageously, the thermal inertia of components (in particular furnaces) allows the reactor to be highly tolerant of a fluctuating energy supply (e.g. a renewable energy supply). Advantageously, systems embodying the invention are relatively small and are suited to integration with a renewable energy source, e.g. wind turbine(s). Embodiments of the invention may for example consume power in the range 50-500 KW, or higher, e.g. up to 10 MW. Preferred Embodiments of the invention are suitable for installation at a renewable energy site, e.g. a wind farm, for utilisation of available unused power at the renewable energy site, but may be scalable for larger capacity use. Use of electric furnace(s) (and/or other electrically powered heating apparatus) also facilitates integration with renewable energy supplies.

Preferred embodiments of the invention are suitable for incorporation into a cascaded energy system in which respective components of the cascaded system (any one of which may comprise an embodiment of the present invention) are provided with energy in a cascaded manner, e.g. wherein a first component may receive energy from a primary energy source (e.g. wind or solar energy source(s)), a second component may receive highest grade waste heat energy, and third component may receive lower grade, secondary or excess waste heat energy, and so on. The cascaded energy system may be configured to cascade energy usage in terms of primary energy and utilisation of waste heat in any suitable manner (e.g. process/component A uses the highest grade waste heat, the waste heat is then used to support process/component B, before finally supporting process/component C).

Further advantageous aspects of the invention will be apparent to those ordinarily skilled in the art upon review of the following description of a specific embodiment and with reference to the accompanying drawings.

### BRIEF DESCRIPTION OF THE DRAWINGS

An embodiment of the invention is now described by way of example and with reference to the accompanying drawings in which like numerals are used to denote like parts and in which:

FIG. 1 is an illustration of an e-fuel production process;

FIG. 2 is a schematic diagram of an e-fuel production system embodying one aspect of the invention;

FIG. 3 is an alternative schematic diagram of the system of FIG. 2 illustrating gas flow around the recirculating reactor; and

FIG. 4 is an alternative schematic diagram of the system of FIG. 3, illustrating a control system.

### Detailed Description of the Drawings

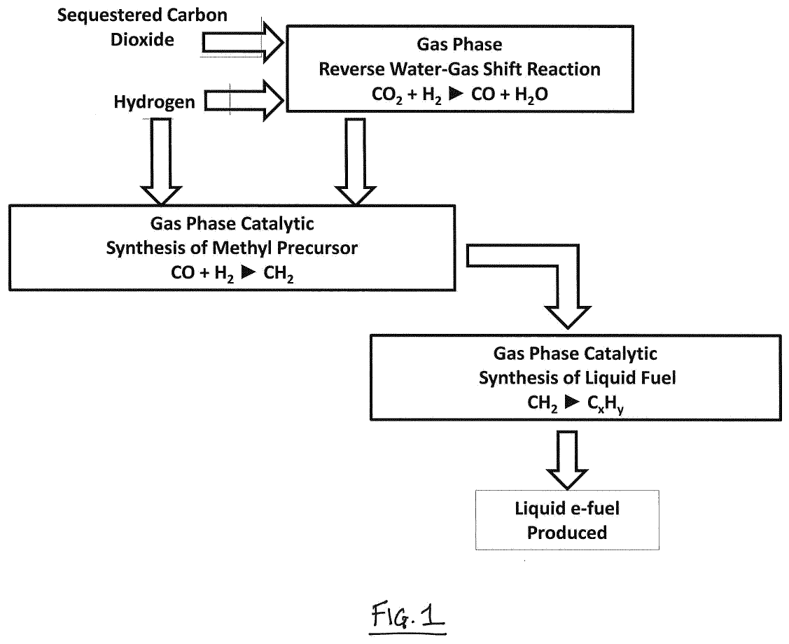

FIG. 1 illustrates a process for synthesizing fuel, in particular e-fuel. The process involves chemically reacting carbon dioxide with hydrogen through the Reverse Water-Gas Shift (RWGS) reaction to produce carbon monoxide and water, shown as a first reaction, Reaction 1, below:

CO₂+H₂ [symbol] CO+H₂O (Reaction 1)

Typically, Reaction 1 involves the use of a catalyst to reduce activation energy and improve processing efficiency. By way of example, suitable catalysts include platinum group metal (PGM) catalysts on a ceramic (or other) support (or washcoat) such as platinum on an alumina/ceria support.

Reaction 1 typically takes place at a temperature of above 600° C., preferably approximately 700° C. There are no particular pressure requirements for the reaction, although overall operating pressure may be dictated by Reactions 2 & 3. The phase of Reaction 1 is dictated by pressure and temperature, which in this case dictates that the reaction is gas phase, preferably accelerated by catalytic reaction on the surface of a suitable catalyst.

The hydrogen and carbon dioxide reactants may be obtained by any conventional means. In preferred embodiments, however, the hydrogen is obtained by a thermochemical cycle in which heat and chemical reactions split water into its hydrogen and oxygen components. The sulphur-iodine cycle is an example of a suitable thermochemical cycle for producing hydrogen (H₂) from water. Preferably, the carbon dioxide is sequestered from the atmosphere, which may be performed by any convenient conventional means, e.g. by chemical direct air capture.

The produced carbon monoxide is mixed with hydrogen to form a gas mixture commonly referred to as syngas (CO+H₂), and a Methyl precursor (e.g. CH₂) is synthesized from the mixture by a second chemical reaction (Reaction 2) commonly known as the Fischer-Tropsch reaction:

CO+H₂ [symbol] CH₂ (Reaction 2)

Typically, Reaction 2 involves the use of a catalyst to reduce activation energy and improve processing efficiency. By way of example, suitable catalysts include iron or cobalt based catalysts on an alumina or silica substrate. Reaction 2 may also produce water.

Reaction 2 typically takes place at approximately 150-300° C. A higher temperature gives faster reaction but also favours shorter carbon chain length. The reaction temperature and reactant concentrations can be tuned to deliver the required carbon chain, e.g. C8 for e-petrol, C12 for e-diesel, and so on. The phase of Reaction 2 is dictated by pressure and temperature, which in this case dictates that the reaction is gas phase, preferably accelerated by catalytic reaction on the surface of a suitable catalyst.

A third chemical reaction, Reaction 3, involves the catalytic synthesis of fuel, preferably e-fuel, from the methyl precursor:

CH₂ [symbol] CₓH_{y} (Reaction 3)

Suitable catalysts include metals such as iron or cobalt, and may comprise a mixed metal catalyst on an alumina or silica support.

Reaction 3 may take place in the same conditions as Reaction 2, e.g. approximately 150-300C. Increasing pressure may drive the reaction forward. Reaction 2 and Reaction 3 may take place together. The phase of Reaction 3 is dictated by pressure and temperature, which in this case dictates that the reaction is gas phase, preferably accelerated by catalytic reaction on the surface of a suitable catalyst.

The reaction product of Reaction 3 is typically gaseous, although this may depend on operating conditions and carbon chain length. For example, C8 e-petrol (boiling point of 125C) will be gaseous but C12 e-diesel (boiling point of 216C) may be liquid depending on the operating temperature.

Any by-products of Reaction 3 are separated from the e-fuel as appropriate.

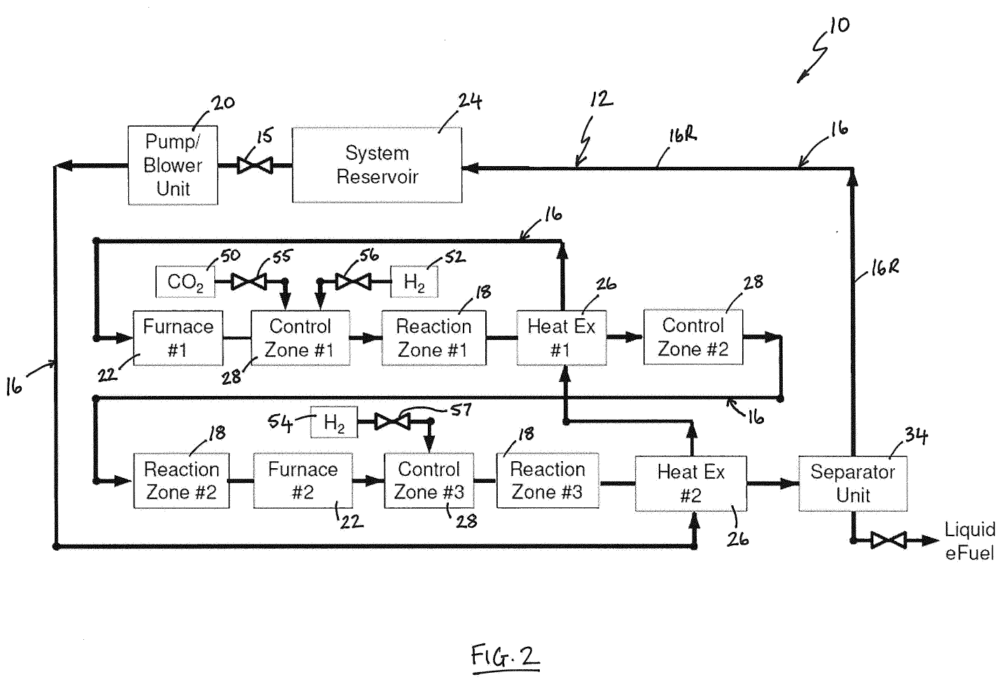

Referring now to FIGS. 2 to 4, there is shown, generally indicated as 10, a synthetic fuel production system, or a fuel synthesizing system, embodying one aspect of the invention. The system 10 is preferably an e-fuel production system. The system 10 includes a reactor 12 and a control system 14 for controlling the operation of the reactor 12. The reactor 12 is intended to cause and control chemical reactions in use and may be described as a chemical reactor. The reactor 12 includes one or more fluid circuits by which fluid (typically gas) may be recirculated within the reactor 12 and, as such, the reactor 12 may be described as a recirculating fluid reactor. In preferred embodiments, the system 10, and in particular the reactor 12, is configured to implement Reactions 1 to 3 outlined above in the recirculating fluid reactor 12, as is described in more detail below.

The reactor 12 comprises a fluid circuit 16 around which fluid is circulated, and advantageously recirculated, during use. The fluid circuit 16 may be of any convenient construction, typically including any one or more of: pipe(s), tube(s), hosing, duct(s) and/or other fluid conduits. These may be formed from any convenient material, e.g. metal or plastics, and may optionally be thermally insulated.

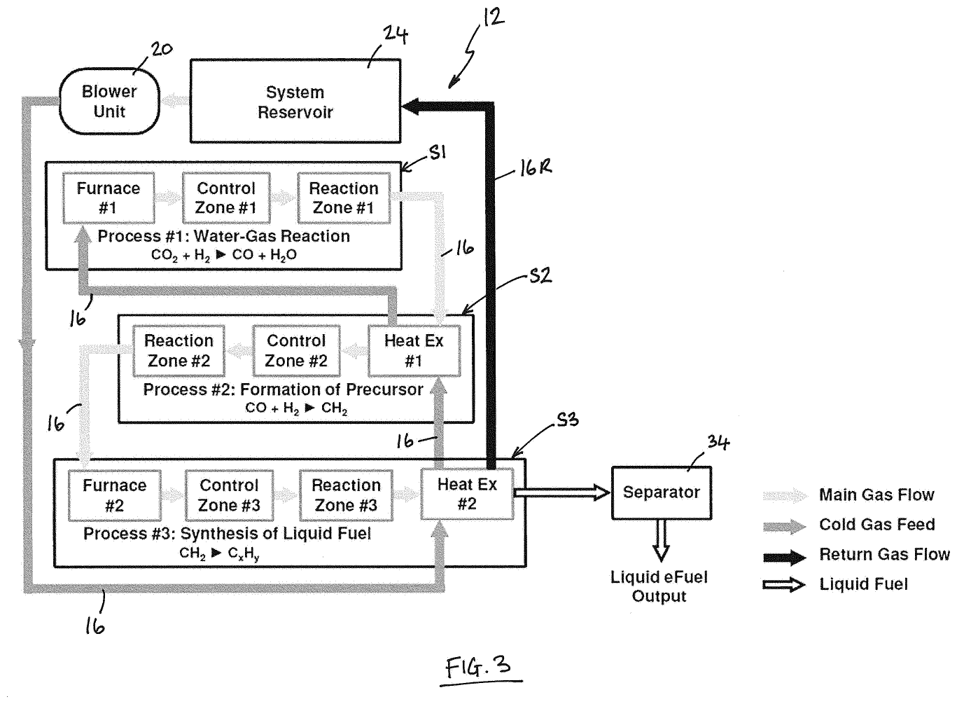

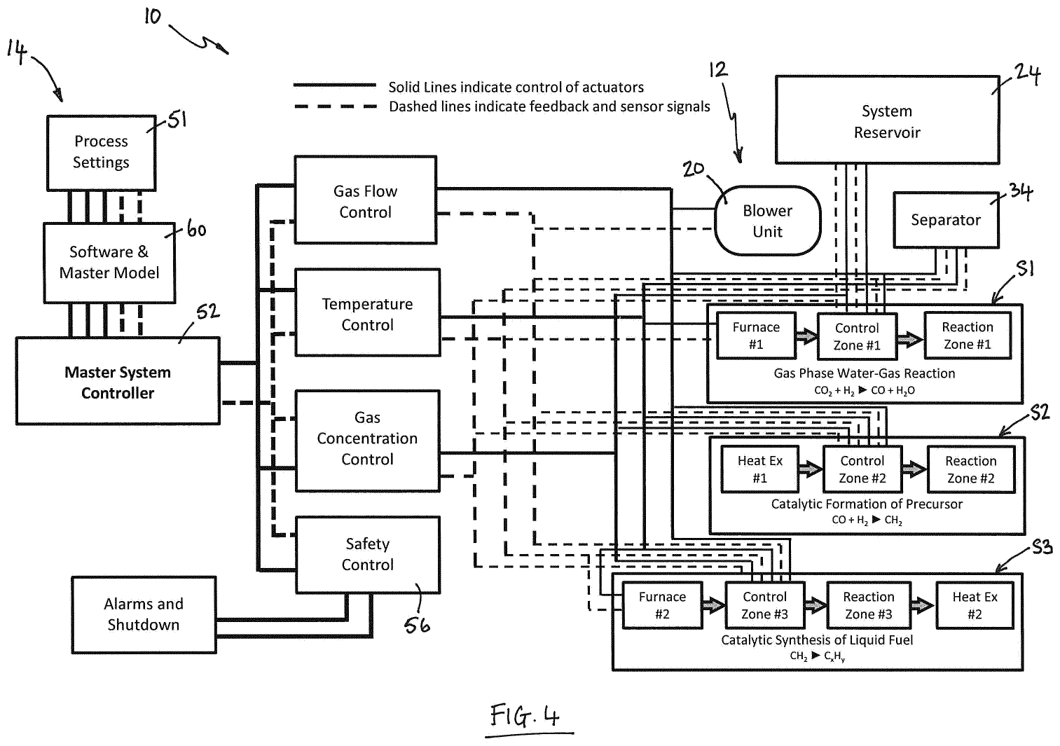

The fluid circuit 16 may include a respective reaction zone 18 for implementing each reaction that is part of the fuel production process. In the illustrated embodiment, the fluid circuit 16 includes a first reaction zone (labelled as Reaction Zone #1 in FIGS. 2 to 4) for implementing Reaction 1 above, a second reaction zone (labelled as Reaction Zone #2 in FIGS. 2 to 4) for implementing Reaction 2,and a third reaction zone (labelled as Reaction Zone #3 in FIGS. 2 to 4) for implementing Reaction 3. However, Reaction 2 and Reaction 3 may conveniently be performed together in the same reaction zone 18. For example, on a macro scale, the Reactions 2 and 3 are likely to happen in the same reaction zone, and the system 10 may be configured to support this. However, on a micro-kinetic scale, Reactions 2 and 3 happen separately, where the precursors form and move to another active site to complete the polymerisation reaction into long chain hydrocarbons. The system 10 may be configured to support the performance of Reactions 2 and 3 separately or together, as required. With reference to FIG. 2, control zone #2 and reaction zone #2 may be omitted, with Reactions 2 and 3 both being performed in reaction zone #3. With reference to FIGS. 3 and 4, processing section S2 may be omitted, with Reactions 2 and 3 both being performed in processing section S3. In preferred embodiments, the reaction zones 18 are arranged in series in the fluid circuit 16. In the illustrated embodiment, the reaction zones 18 are arranged in series and in sequence such that fluid exiting reaction zone #1 is delivered (typically indirectly) to reaction zone #2, and that fluid exiting reaction zone #2 is delivered (typically indirectly) to reaction zone #3. In embodiments where reaction zone #2 is omitted, the reaction zones 18 are arranged in series and in sequence such that fluid exiting reaction zone #1 is delivered (typically indirectly) to reaction zone #3.

Each reaction zone 18 may take any suitable form, for example comprising a chamber or vessel incorporated into the fluid circuit 16, or being a part of a conduit that forms the fluid circuit. Each reaction zone 18 is in fluid communication with the fluid circuit 16 such that fluid may be delivered to and from the reaction zone 18 during use.

The reactor 12 typically includes, or is connected to, at least one fluid reservoir 24 for storing quantities of fluid, typically gas, and which may also storing energy (i.e. by storing fluid at a temperature that is elevated compared to the fluid in the circuit). In preferred embodiments, the reactor 12 includes reservoir 24 for storing a carrier gas (typically comprising nitrogen and/or other suitable gas(s) (e.g. inert gas(es)), typically mixed with recycled gas(es) (which may include unused reactants and/or by-products from the reactions) delivered to the reservoir 24 by a return part 16R of the fluid circuit 16). The reservoir 24 is conveniently included in, or connected to, the fluid circuit 16 and may be located upstream of reaction zone #1, or at any other convenient location in the circuit 16. In alternative embodiments, a carrier gas is not used and the reactants and reaction products may be circulated around the reactor as applicable without a carrier gas. Optionally, reservoir 24 may be omitted.

The reactor 12 typically includes fluid driving means 20 for causing fluid (typically gas) to flow around the fluid circuit 16. The fluid driving means 20 may take any conventional form, e.g. comprising one or more fans or blowers (for example including axial fans, propeller fans, centrifugal (radial) fans, mixed flow fans and cross flow fans), pumps (e.g. centrifugal pumps or positive displacement pumps), compressors and/or turbines or other fluid driving device. The fluid driving means 20 is preferably controllable to control the flow, and in particular the flow rate, of fluid around the fluid circuit 16. Advantageously, the fluid driving means 20 is operable to control the pressure of the fluid in the fluid circuit. The system may control fluid pressure as a tuning parameter for the fuel production: temperature, pressure and reactant mix are typically the control parameters.

Advantageously, the system 10 may control the pressure at multiple locations in the reactor 12, typically using one or more fluid driving devices (not illustrated) at or associated with those locations in order to optimise individual Reactions 1, 2 and/or 3. Flow of fluid around the circuit 16 may also be controllable using one or more valves 15.

The reactor 12 includes heating means 22 for controlling the temperature of fluid in the circuit 16, particularly in the reaction zones 18. In typical embodiments, the heating means comprises one or more furnace, boiler and/or other heating device. In the illustrated embodiment, a first furnace (labelled as Furnace #1 in FIGS. 2 to 4) is included in or associated with reaction zone #1, and a second furnace (labelled as Furnace #2 in FIGS. 2 to 4) is included in or associated with reaction zone #3. In alternative embodiments, other heating devices may be used instead of furnaces. The heating means 22 may for example comprise one or more instance of any one or more of the following heating devices: chemical or gas furnaces (e.g. a propane or natural gas furnace) or electrical furnaces (e.g. an infra red furnace, electrical tube furnace or flat bed furnace) or any other convenient heating device including electrical heater(s), infra red heater(s), gas heater(s) and/or heat lamp(s) (e.g. quartz or tungsten heat lamps). Use of electrically powered furnaces and/or other electrically powered heating devices is preferred as it facilitates integration with a renewable energy supply. The heating means 22 are preferably controllable to control and/or modulate the temperature of the fluid in the respective part of the circuit 16 and so to control and/or modulate a base temperature in the respective reaction zone 18 and/or control the temperature of the reactants as required. The heating means may be connected or coupled to the fluid circuit in any suitable manner.

In some embodiments, the reactor, or more particularly the fluid circuit 16, may be coupled to external heating means (not shown) configured to provide heat energy to the reactor, e.g. for controlling the temperature of fluid in the fluid circuit 16, the heat energy advantageously being waste heat energy. The external heating means may comprise an external apparatus or system configured to perform an industrial process, e.g. cement production, glass production, steel production and/or any other waste heat producing industrial process. The external heating means may be coupled to the reactor, or more particularly the fluid circuit 16, by any suitable conventional coupling means (e.g. one or more heat exchanger) and/or via any convenient heat exchanging medium (e.g. steam) in order that heat energy, preferably waste heat energy, may be transferred to the reactor/fluid circuit. For example, in the illustrated embodiment, one or more external heating means may be coupled to the fluid circuit 16 at the locations of Furnace #1 and/or Furnace #2 (as well as or instead of the furnaces).

The reactor 12 advantageously includes one or more heat exchanger 26 to improve the efficiency of the reactor 12 in particular with respect to maintaining desired fluid temperatures in the reactor 12 energy efficiently. The heat exchangers 26 may be gas to gas type, gas to liquid type or liquid to liquid type as appropriate. In the illustrated embodiment, a first heat exchanger (labelled as Heat Ex #1 in FIGS. 2 to 4) is included in the fluid circuit 16 and is configured to regenerate heat from reaction zone #1. Advantageously, the first heat exchanger is configured to use heat from the exit fluid from reaction zone #1 to heat fluid being delivered (indirectly) to reaction zone #1, the heat exchange process simultaneously cooling the reaction zone #1 exit fluid. In the illustrated embodiment, a second heat exchanger (labelled as Heat Ex #2 in FIGS. 2 to 4) is included in the fluid circuit and is configured to regenerate heat from reaction zone #3. Advantageously, the second heat exchanger is configured to use heat from the exit fluid from reaction zone #3 to heat fluid being delivered (indirectly) to reaction zone #1, the heat exchange process simultaneously cooling the exit fluid from reaction zone #3.

In preferred embodiments, the reactor 12 includes a plurality of control zones 28. Each control zone 28 is incorporated into the fluid circuit 16 at a respective location. Any one or more of the control zones 28 may be equipped to measure at least one aspect of the reactor's operation. Each control zone 28 may be configured to measure one or more characteristic, or parameter, of the fluid at the respective location in the respective fluid circuit 16 into which it is incorporated. As is described in more detail hereinafter, each control zone 28 may for example be configured to measure any one or more of the following fluid characteristics: flow rate, temperature, chemical composition, pressure, and may include any suitable conventional measurement device(s) for this purpose. Any one or more of the control zones 28 may be configured to control one or more characteristic of the fluid in the fluid circuit 16, e.g. the fluid flow rate, temperature, pressure and/or chemical composition, and/or to divert, direct or otherwise control the flow of the fluid, e.g. to a vent or drain to another component of the reactor 12. To this end, each control zone 28 may include one or more control devices, e.g. one or more valves 15, fluid injectors or fluid mixing devices. Any one or more of the respective control device(s) may be located at the respective control zone 28, in which case the control zone 28 controls the relevant fluid characteristic directly in its own locality. Alternatively, any one or more of the respective control device(s) may be located remotely from the respective control zone 28, in which case the control zone 28 controls the relevant fluid characteristic in one or more locations in the fluid circuit(s) remote from the control zone 28 itself. In such cases the control zone 28 may be said to include the control device in that it controls the operation of the control device.

In preferred embodiments, any one or more of the control zones 28 may be configured to monitor and control the introduction of one or more fluids, typically gas(es), into the fluid circuit 16 (e.g. to control reactant levels and/or concentrations). To this end, each such control zone 28 may comprise one or more fluid injectors and/or valves 15. Each fluid injector may take any conventional form, typically comprising one or more valves and conduit(s) connected to one or more fluid sources, e.g. a canister, a compressor and/or one or more other vessel or reservoir, usually pressured fluid sources. Each fluid source may contain a single fluid or a mixture of two more fluids, depending on the application and the tasks being performed by the respective control zone. Each fluid injector is operable to selectable inject one or more fluids into the respective fluid circuit(s) via one or more fluid inlets (not shown). Conveniently, the fluid inlet(s) are located at the respective control zone 28, although they may alternatively or additionally be located elsewhere in the fluid circuit(s). Conveniently, each fluid injector is located at the respective control zone 28, although they may alternatively or additionally be located elsewhere in the fluid circuit(s). Optionally, one or more fluid injectors (not shown) may be provided for injecting fluid(s) into the reservoir(s).

In order to communicate with other components of the system 10, including for example remote analyser(s) and/or a control system, each control zone 28 may include a communications system including one or more wired and/or wireless communications devices as required.

The control zone 28 typically includes an enclosure in which at least some of its components are housed as is convenient. The enclosure may for example comprise a chamber incorporated into the circuit 16, or a chamber to which the circuit 16 is connected or passes through, or may comprise a part of one or more conduits that form the circuit 16.

The reactor 12 includes at least one separating apparatus for separating the products produced by the reactions implemented in the reaction zones 18. In preferred embodiments, the reactor 12 includes separator 34 which is configured and arranged to separate the synthesized fuel from any other products of Reaction 3 and/or the carrier gas, as applicable. The separator 34 may take any conventional form, and may comprise any suitable conventional type(s) of separation means, to suit the method(s) by which the relevant products can be separated, e.g. condensation, distillation or liquid/liquid gravitic or gravitmetric separation, membrane separation and/or selective adsorption. In preferred embodiments, the fuel is produced in liquid form (conveniently having been condensed by the second heat exchanger Heat Ex #2). The separator 34 may be required to perform condensation since Heat Ex #2 may not facilitate complete condensation. Separator 34 is configured to separate the desired fuel products from reaction zone 3, i.e. long chain hydrocarbons, from other products e.g. water and unconverted reactants.

In preferred embodiments, the fluid circuit 16 is configured to form a loop around which fluid can be recirculated, the reaction zones 1, 2 & 3 being provided in series and in sequence in the loop. Advantageously, respective portions of the fluid circuit 16 are brought together (i.e. are sufficiently close to enable heat exchanging) at at least one location in the loop to facilitate heat exchanging between the respective circuit portions. The respective circuit portions may cross each other at such heat exchanging locations (as illustrated) but need not necessarily do so. In preferred embodiments, respective circuit portions are brought together in Heat Ex #1, which is located downstream of reaction zone #1, preferably immediately downstream of reaction zone #1, e.g. at the fluid exit of reaction zone #1. The circuit portions brought together at Heat Ex #1 may be the circuit portion carrying the products of reaction zone #1 and, typically, the circuit portion that carries fluid to furnace #1. In preferred embodiments, respective circuit portions are brought together in Heat Ex #2, which is located downstream of reaction zone #3, preferably immediately downstream of reaction zone #3, e.g. at the fluid exit of reaction zone #3. The circuit portions brought together at Heat Ex #3 may be the circuit portion carrying the products of reaction zone #3 and, preferably, the circuit portion that carries fluid to furnace #1, more preferably the circuit portion that carries fluid to Heat Ex #1 for delivery to furnace #1.

The system 10 includes a control system 14 for controlling and/or monitoring the operation of the system components, including, as required, the reaction zones 18, control zones 28, valves, fluid drivers 20, furnaces 22 and separator 34 and any other controllable device (e.g. fluid injectors, sensors and so on). The control system 14 typically comprises a master controller 52 which is typically implemented by one or more suitably programmed or configured hardware, firmware and/or software controllers, e.g. comprising one or more suitably programmed or configured microprocessor, microcontroller or other processor, for example an IC processor such as an ASIC, DSP or FPGA (not illustrated).

In preferred embodiments the control system 14 communicates control information to other components of the system 10, for example the control zones 28, valves, fluid drivers 20 and/or furnaces 22 in order to implement Reactions 1, 2 and 3. Process settings may be received via a process settings interface unit 51. The process settings may specify environmental conditions, for example in relation to temperature(s), flow rate(s), and/or pressure(s), and/or reactant levels (and/or concentrations) for the reaction zones 18. The control system 14 may also receive feedback information from other components of the system 10, for example the control zones 28, sensors, measurement devices, valves, fluid drivers 20 and/or furnaces 22, in response to which the control system 14 may issue control information to one or more relevant system components. To this end the control system 14 may perform analysis of the measurements or other information provided by the control zones 28. This analysis may be carried out automatically in real time by the control system 14. Alternatively, or in addition, analysis of the system measurements and performance may be made by an operator in real time or offline. The operator may make adjustments to the operation of the system 10 by providing control instructions via interface unit 51.

In preferred embodiments, the reactor 12 is controlled to provide uniformity in the reaction zones, or in the reactor bed, particularly temperature uniformity, and this may be achieved through zonal controlled cooling and/or heating.

A safety controller 56 may be provided, which may receive alarm signals from one or more alarm sensors (not shown), e.g. gas sensors or leak detectors or emergency stops that may be included in the system 10, and provide alarm information to the master controller 52 based on the alarm signals received from the alarm sensors.

In preferred embodiments, the control system 14, and more particularly the master controller 52 is configured to implement system modelling logic, e.g. by supporting mathematical modelling software or firmware 60, for enabling the control system 14 to mathematically model the behaviour of the system 10, and in particular of the reactor 12, depending on the process settings and/or on feedback signals received from one or more system components during operation of the system 10.

Optionally, the control system 14 is configured to implement Model Predictive Control (MPC). Using MPC, the control system 14 causes the control action of the control zones 28 to be adjusted before a corresponding deviation from a relevant process set point actually occurs. This predictive ability, when combined with traditional feedback operation, enables the control system 14 to make adjustments that are smoother and closer to the optimal control action values than would otherwise be obtained. A control model for the system 10 can be written in Matlab, Simulink, or Labview by way of example and executed by the master controller 52. Advantageously, MPC can handle MIMO (Multiple Inputs, Multiple Outputs) systems.

The control system 14 may include an artificial intelligence (AI) based model controller configured to optimize operation of the system 10 in real time in order to making best use of available energy, reactant levels and so on.

Advantageously, one or more parts of the reactor 12 may be configured in a modular manner to facilitate modular construction and transportation of the reactor 12 (or any part thereof), and/or to facilitate modular scaling of the reactor 12 or any part thereof. For example, each reaction zone 18 may be provided in a respective reactor module, which may be referred to as a sub-reactor. Advantageously, each reactor module is configured to support modular scaling of the respective reaction zone 18. For any given reaction zone 18, one or more instance of the respective type of reactor module may be provided (and modularly interconnected as required) in order to perform the respective reaction(s). The selected number of instances of reactor module that are used may depend on one or more desired operating parameter (e.g. any one or more of: energy usage, available energy, reactant usage, reactant availability, reaction product production rate, and so on) of the relevant application. As a result, the reactor 12, or any modular part thereof, may be scaled as suits the application. In preferred embodiments therefore, the reactor 12 comprises one or more chemical sub-reactors built in modules for easier fabrication/manufacture and transport. Furthermore, the reactor output may be sized, or scaled, based on the number of modules provided for each reaction, rather than solely through the size of individual reactors. This adds the benefit of extended turndown ratio for the reactor. Additionally, ancillary equipment (e.g. valve(s), pump(s) and/or heater(s)) and/or pre-and post-processing steps (e.g. fractional distillation) can be included in the modules as required.

The size of the reactor 12, in particular in terms of its power consumption, may vary to suit the application. Advantageously, sizing or scaling of the reactor 12 is supported by the preferred modularity of the reactor 12, or at least part(s) thereof. For example reactors embodying the invention may be designed with power consumption ranges of up to 200 KW, up to 500 KW, up to 1 MW, up to 2 MW, up to 5 MW or up to 10 MW, as required.

The preferred embodiment is now described in more detail. The reservoir 24 comprises a suitable vessel, e.g. pressure vessel, for storing the carrier gas and any other gas that may be recycled to the reservoir 24 by the return part 16R of the fluid circuit 16. The reservoir 24 includes at least one inlet for receiving the relevant gas(es) from the recirculating, or return, part 16R of the circuit 16, and typically also from an external source of the carrier gas (e.g. for initially charging the reservoir 24 with carrier gas and for topping up the carrier gas if required). The reservoir 24 may include or be associated with means for controlling the flow of carrier gas from the reservoir 24 into the fluid circuit 16, e.g. a valve 15, fluid injector or other fluid control device. A valve, e.g. a non-return valve, may be provided to control the return of fluid to the reservoir.

The reservoir 24 may comprise any one or more of the following components as required and as applicable: a heating device; a cooling device; pressure measurement device(s); temperature measurement device(s); isolation valve(s); pressure relief valve(s); level measurement device(s), each of which may be controlled by the control system 14 and/or provide information to the control system 14, e.g. to ensure that the carrier gas is stored in the desired conditions, and/or to control the flow of carrier gas into and/or out of the reservoir 24. Typically, indications of fluid level, pressure and/or temperature are provided to the control system 14 by the reservoir 24. The reservoir stores the carrier gas that circulates around the fluid circuit 16 in use, carrying the reactants for, and reaction products of, Reactions 1, 2 and 3 around the fluid circuit 16 as required, in particular to and from the respective reaction zones 18. Advantageously, the reservoir 24 may provide a buffer to allow variable process rates in the reaction zones and/or elsewhere in the reactor to be accommodated.

The carrier gas (and any other gases carried with it, e.g. reactants and/or reaction products as applicable) is driven around the fluid circuit by the fluid drive means 20 in the preferred form of a pump(s), compressor(s) and/or blower(s), e.g. a high speed centrifugal blower. Preferably, the fluid drive means 20 includes variable speed drive that is controllable by the control system 14. The fluid drive means 20 may also include one or more flow measurement device for providing flow information to the control system 14.

In the illustrated embodiment, the reactor 12 is configured to implement Reactions 1, 2 and 3 in a respective processing section (labelled as S1, S2, S3 in FIGS. 3 and 4) of the reactor 12. In alternative embodiments, processing section S2 may be omitted and Reactions 2 and 3 may be performed together in processing section S3. Each processing section S1, S2, S3 comprises a respective control zone 28 (labelled as control zone #1, control zone #2 and control zone #3 in

FIGS. 2 to 4) and a respective one of the reaction zones 18 (labelled as reaction zone #1, reaction zone #2 and reaction zone #3 in FIGS. 2 to 4). Each control zone 28 is operable (by control system 14 in preferred embodiments) to control one or more of the characteristics of the fluid (typically gas) in the respective reaction zone 18. Preferably, each control zone 28 is located upstream of the respective reaction zone 18, more preferably immediately upstream of the respective reaction zone 18, e.g. at a fluid inlet of the respective reaction zone 18. Each control zone 28 is equipped with one or more sensor/measurement device and/or one or more control device (e.g. valve and/or fluid injector) to allow it to monitor and/or control the relevant characteristic(s) of the fluid in the respective reaction zone 18.

In preferred embodiments, each control zone 18 (i.e. control zone #1, control zone #2 and control zone #3) is operable to monitor and/or control the fluid temperature, fluid pressure, fluid flow rate and fluid composition in the respective reaction zone 18. To this end, each control zone 28 may comprise any one or more of the following components: flow control and/or pressure regulating valve(s) with remote actuator(s) and/or mass flow controller(s) or other fluid injector(s); fluid separating device(s);

fluid driving device(s); flow measurement device(s); pressure measurement device(s); temperature measurement device(s), fluid level and/or composition measurement device(s), each of which may be controlled by the control system 14 and/or provide information to the control system 14 as required. For example, each control zone 28 may send information to the control system 14 indicating measured flow rate, fluid pressure, fluid temperature and/or fluid composition as required.

Each control zone 28 may receive control signals from the control system 14 for operating the relevant valve(s) and/or fluid injector(s) to control fluid flow rate, fluid pressure and/or fluid composition as required. Fluid temperature is typically controlled by controlling the, or each, heating device associated with the respective reaction zone 18, e.g. furnace #1 and furnace #2 in the illustrated embodiment.

The reactor 12 is configured to receive hydrogen gas (H₂) and carbon dioxide gas (CO₂) from a respective suitable source, e.g. comprising a tank, canister or other suitable reservoir. Preferably, the hydrogen is obtained by a thermochemical cycle in which heat and chemical reactions split water into its hydrogen and oxygen components, and the carbon dioxide is sequestered from the atmosphere. The H₂ and CO₂ may be introduced into the fluid circuit 16 at any desired location(s), typically from a respective reservoir 50, 52, 54 (reservoirs 52, 54 may be the same reservoir or different reservoirs as is convenient). In preferred embodiments, H₂ and CO₂ are introduced into the fluid circuit 16 at control zone #1 to provide reactants for Reaction 1, although the H₂ and CO₂ may alternatively be introduced into the fluid circuit elsewhere upstream of reaction zone #1. Optionally, H₂ may also be introduced into the circuit 16 as a reactant for Reaction 2 at a location between reaction zone #1 and reaction zone #2, e.g. at control zone #2 (not illustrated but may be the same as shown for control zones #1 and #3), although the arrangement may be such that the H₂ introduced at control zone #1 or otherwise upstream of reaction zone #1 may be sufficient for reaction #2 in reaction zone #2. Water or other unwanted products from reaction zone #1 may optionally be separated from the desirable reaction products and removed via a drain prior to reaction zone #2 or reaction zone #3.

The amount of H₂ that is present for Reaction 3 can affect which long chain hydrocarbon, and therefore fuel, that is synthesized from the methyl precursor. While H₂ may already be present in the fluid delivered to reaction zone #3 from the upstream reaction zone(s) 18, it is preferred that the system 10 can control the amount of H₂ that is present for Reaction 3 in order to control the type (e.g. chemical composition) of the hydrocarbon or fuel that is produced. Therefore, H₂ may also be introduced into the circuit 16 for Reaction 3, e.g. at control zone #3 or otherwise upstream of reaction zone #3 but downstream of reaction zone #1 and reaction zone #2 if present, to support the formation of the relevant long chain hydrocarbons from the methyl precursor. A respective fluid inlet device 55, 56, 57 (e.g. comprising a valve and/or fluid injector) is provided for controlling the flow of H₂ or CO₂ into the fluid circuit 16, preferably under the control of control system 14.

Each reaction zone 18 typically comprises a reaction vessel or conduit (e.g. a containment or pressure vessel or tube), and may further include any one or more of the following components: flow control and/or pressure regulating valve(s) with remote actuator(s) and/or mass flow controller(s) or other fluid injector(s); flow measurement device(s); pressure measurement device(s); temperature measurement device(s), fluid level and/or composition measurement device(s), each of which may be controlled by the control system 14 and/or provide information to the control system 14 as required. For example, each reaction zone 18 may send information to the control system 14 indicating measured flow rate, fluid pressure, fluid temperature and/or fluid composition as required. Each reaction zone 28 may receive control signals from the control system 14 for operating the relevant valve(s) and/or fluid injector(s) to control fluid flow rate, fluid pressure and/or fluid composition as required. As such, each reaction zone 18 may be said to comprise a control zone.

Processing sections S1, S3 each includes a respective heating apparatus, preferably comprising a furnace 22 (labelled as furnace #1 and furnace #2 in FIGS. 2 to 4). The heating apparatus typically comprises a containment or pressure flow conduit for a thermal mass for storage and transfer of heat. The heating apparatus may include any conventional heating device(s), e.g. electrical, gas or liquid fuel combustion or heat exchange type(s). In preferred embodiments, each furnace 22 comprises a high thermal inertia electric furnace. More generally an electrically powered furnace is preferred. Alternatively, a combustion based heater or other heating device(s) may be used to perform the required heating. Each heating apparatus may include any one or more of the following components: flow control and/or pressure regulating valve(s) with remote actuator(s) and/or mass flow controller(s) or other fluid injector(s); flow measurement device(s); pressure measurement device(s); temperature measurement device(s), fluid level and/or composition measurement device(s), each of which may be controlled by the control system 14 and/or provide information to the control system 14 as required. Preferably, furnaces #1 and #2 are located upstream of the respective control zone 28, more preferably immediately upstream of the respective control zone 28, e.g. at a fluid inlet of the respective control zone 28.

In preferred embodiments, heat exchanger Heat Ex #1, which may be part of processing section S1, is located downstream of the fluid exit of reaction zone #1, preferably at the fluid exit of reaction zone #1, such that fluid exiting reaction zone #1 passes through Heat Ex #1. In addition, Heat Ex #1 is arranged to receive fluid being delivered to processing section S1 such that a heat exchange process is performed between fluid being delivered to, and fluid exiting from, the processing section S1. Heat exchanger Heat Ex #2, which may be part of processing section S3, is located downstream of the fluid exit of reaction zone #3, preferably between the fluid exit of reaction zone #3 and the separator 34, such that fluid exiting reaction zone #3 passes through Heat Ex #2 (preferably on its way to the separator 34). In addition, Heat Ex #2 is arranged to receive fluid being delivered to processing section S1 such that a heat exchange process is performed between fluid being delivered to processing section S1 and fluid exiting from reaction zone #3 (or processing section S3). The preferred arrangement is such that the fluid received by Heat Ex #1 for delivery to section S1 is received from Heat Ex #2, although the reverse arrangement may alternatively be implemented. In either case, it is preferred that the fluid being delivered to processing section S1 passes through both Heat Ex #1 and Heat Ex #2 in order that the fluid is heated by heat exchange with the exit fluid from both reaction zone #1 and reaction zone #2 (or processing section S1 and processing section S3). In alternative embodiments, one or other or both of the heat exchangers Heat Ex #1, Heat Ex #2 may be omitted. Alternatively or in addition a third heat exchanger (not shown) may be provided at the fluid exit of reaction zone #2/processing section S2 and arranged to perform a heat exchange operation between fluid exiting reaction zone #2/processing section S2 and fluid being delivered to processing section S1. The heat exchangers may comprise any suitable type, typically a gas-gas or gas-liquid heat exchanging device. Each heat exchanger may include any one or more of:

temperature measurement device(s); pressure measurement devices(s), each of which may be controlled by the control system 14 and/or provide information to the control system 14 as required.

The reaction product(s) from reaction zone #3 are delivered to the separator 34, which is configured to separate the fuel from the carrier gas, any by-products and/or any unused reactants. The separator 34 may comprise a condenser for condensing the fuel and/or any conventional liquid-gas separation apparatus. Separator 34 may also comprise a gravimetric type device to separate different hydrocarbon products. The separator 34 may include a pressure vessel. The separator 34 may include any one or more of: temperature measurement device(s); pressure measurement devices(s), each of which may be controlled by the control system 14 and/or provide information to the control system 14 as required.

In preferred embodiments the processing sections S1, S2 (if present), S3 are connected in series by the fluid circuit 16 such that the reaction product(s) from reaction zone #1 are delivered to reaction zone #2, and the reaction product(s) from reaction zone #2 are delivered to reaction zone #3, or such that the reaction product(s) from reaction zone #1 are delivered to reaction zone #3 in embodiments where reaction zone #2 is omitted. The reaction product(s) from reaction zone #3 are delivered to the separator 34. The carrier gas is delivered to an inlet of the first processing section S1 and is directed through each processing section S1, S2 (if present), S3 in turn by the fluid circuit 16, carrying with it the reaction product(s). The fluid circuit 16 is configured to form a loop such that the carrier gas may be recirculated through the processing sections S1, S2 (if present), S3. In each processing section S1, S2 (if present), S3, one or more characteristics of the fluid are controlled to implement the respective reaction (Reaction 1, Reaction 2, Reaction 3) in the respective reaction zone 18. In particular any combination of any one or more of the following fluid characteristics may be controlled as required: fluid flow rate, fluid temperature, fluid composition and/or fluid pressure. Controlling the fluid characteristics is performed by the control system 14 in co-operation with the components of the respective processing section S1, S2 (if present), S3 as required.

Operation of the preferred embodiment is now described in more detail. A quantity of carrier gas mix is stored in reservoir 24, typically at less than 100° C. The carrier gas mix (typically comprising a mix of nitrogen (or other inert gas) and/or recycled gases) is cycled around the fluid circuit 16 under the action of the drive means 20 (being drawn from and returned to reservoir 24 in the illustrated example). The carrier gas is delivered to the first processing section S1 via the heat exchangers Heat Ex #1 and Heat Ex #2. Heat Ex #2 regenerates heat from the exit fluid of reaction zone #3 to heat the carrier gas. Typically, Heat Ex #2 raises the temperature of the carrier gas to approximately 200° C., typically capturing at least 60% of the heat energy of the fluid exiting reaction zone #3. Heat

Ex #1 regenerates heat from the exit fluid of reaction zone #1 to further heat the carrier gas. Typically, Heat Ex #1 raises the temperature of the carrier gas to approximately 500° C., typically capturing at least 50% of the heat energy of the fluid exiting reaction zone #1. Furnace #1 heats the received fluid (gas) to a temperature suitable for implementing Reaction 1. Typically, Furnace #1 heats the incoming carrier gas to 700° C. or approximately 700° C.

H₂ and CO₂ are introduced into the circuit 16 at control zone #1. Control zone #1 monitors and controls the fluid (gas) flowing through it such that the fluid characteristics (typically gas temperature, gas composition and gas flow rate) are suitable for implementing Reaction 1 in reaction zone #1. The gas exiting control zone #1 (the carrier gas mixed with CO₂ and H₂) is provided to reaction zone #1 as a reactant. Reaction 1 is implemented in reaction zone #1 to produce its reaction products, including CO. Optionally, reaction zone #1 monitors and controls the fluid (gas) flowing through it such that the fluid characteristics (typically gas temperature, gas composition and gas flow rate) are suitable for implementing Reaction 1 in reaction zone #1.

The reaction products from reaction zone #1, mixed with the carrier gas, are delivered to control zone #2 (or control zone #3 if control zone #2 is not present) via Heat Ex #1. Heat Ex #1 cools the exit gas from reaction zone #1 (by heat exchange with the carrier gas received by Heat Ex #1 as described above), preferably to a temperature that is suitable for implementing Reaction 2 in reaction zone #2. Typically, Heat Ex #1 cools the exit gas to 300° C. or approximately 300° C.

H₂ is optionally introduced into the circuit 16 at control zone #2. Excess water is optionally removed from the circuit at or before control zone #2. Control zone #2 monitors and controls the fluid (gas) flowing through it such that the fluid characteristics (typically gas temperature, gas composition and gas flow rate) are suitable for implementing Reaction 2 in reaction zone #2. The gas exiting control zone #2 (which comprises the carrier gas mixed with CO and H₂) is provided to reaction zone #2 as a reactant. Reaction 2 is implemented in reaction zone #2 to produce its reaction products, which comprises the methyl precursor or other fuel precursor. Optionally, reaction zone #2 monitors and controls the fluid (gas) flowing through it such that the fluid characteristics (typically gas temperature, gas composition and gas flow rate) are suitable for implementing Reaction 2 in reaction zone #2. In alternative embodiments, control zone #2 is omitted and Reaction 2 is performed in reaction zone #3 with Reaction #3. In such cases, the description provided in relation to control zone #2 and reaction zone #2 may be applied to control zone #3 and reaction zone #3.

For this combination of reactions, Reaction 2 and Reaction 3 may be represented as:

CO+(1+a/2).H₂→CHₐ+H₂O

which is the overall reaction for producing a synthesized fuel, e.g. a liquid e-fuel, represented in the short form of CHₐ. The value of parameter a determines which hydrocarbon is produced, and may be controlled by controlling the amount of H₂ that is present (for Reaction 3 in particular). For example e-petrol is mostly C₈H₁₈ but can be represented as CHₐ where parameter a has the value 2.25 in this example.

The exit fluid from reaction zone #2 (or reaction zone #1 if reaction zone #2 is not present) is delivered to furnace #2. Furnace #2 heats the received fluid (gas) to a temperature suitable for implementing Reaction 3 (or Reactions 2 and 3 as applicable). Typically, Furnace #2 heats the incoming gas to 150-300° C. The heated fluid is delivered to control zone #3. Control zone #3 monitors and controls the fluid (gas) flowing through it such that the fluid characteristics (typically gas temperature, gas composition and gas flow rate) are suitable for implementing Reaction 3 (and optionally also Reaction 2) in reaction zone #3. The gas exiting control zone #3 (the carrier gas mixed with the fuel precursor) is provided to reaction zone #3 as a reactant. Reaction 3 is implemented in reaction zone #3 to produce its reaction products, including the e-fuel in gas form. Optionally, reaction zone #3 monitors and controls the fluid (gas) flowing through it such that the fluid characteristics (typically gas temperature, gas composition and gas flow rate) are suitable for implementing Reaction 3 in reaction zone #3.

Reaction 3 may be represented as:

X.CHₐ+B.H₂→CₓH_{y}

Where typically X=8 and a=2. If the synthesized fuel is purely octane, then y=18 and B=1. The process will not necessarily yield just octane but typically a mix with some other hydrocarbons (e.g. C7 and C9) so these values are approximate and depend on the blend. The ratio of carbon to hydrogen is controlled within reaction zone #3, typically along with other operating conditions including pressure and/or temperature, to ensure that the desired CₓH_{y} products are produced.

The reaction products from reaction zone #3, typically mixed with the carrier gas, are delivered to the separator 34 via Heat Ex #2. Heat Ex #2 cools the exit gas from reaction zone #3 (by heat exchange with the carrier gas received by Heat Ex #2 as described above), typically reducing the temperature to less than 100° C. The separator 34 is configured to separate the synthesized fuel from the carrier gas, any by-products and/or any unused reactants. Heat Ex #2 may be configured to condense the fuel received from reaction zone #3, in which case the separator 34 may comprise any suitable conventional liquid-gas separating apparatus. Alternatively, the fuel may be provided to the separator 34 in gaseous form, in which case the separator 34 may comprise a condenser and optionally also any suitable conventional liquid-gas separating apparatus. The separated fuel may be collected from the system 10 by any convenient collection means and/or outlet means, and/or may be stored in any convenient storage means, e.g. tank (not shown), typically under the control of a valve 15 or other fluid outlet control means. The separated carrier gas (which may be mixed with gaseous by-products from the reactions and/or unused reactants) is recycled to the reservoir 24 by the return part 16R of the circuit 16.

Optionally, product(s), or other substance(s) that are recycled for use in Reaction 1, i.e. recycled via the return part 16R of the circuit 16 in the illustrated embodiment, are recycled separately from each other. To this end, the return part of the circuit may comprise multiple conduits, or a conduit configured to carry multiple fluids (or gases in particular) separately, e.g. using one or more separating membranes. The reservoir 24 may be configured to store each returned substance separately, or more than one reservoir may be provided as is convenient. For example, in preferred embodiments, return line 16R does not contain a mixture of gas products, it rather contains multiple streams of individual species separated through membrane separation. The separator 24 may be provided with any suitable conventional type(s) of separation means for performing the required separation (e.g. it may comprise condenser(s), membrane(s), selective adsorption means and so on).

It will be apparent that the operation described above regenerates, or recycles, heat, reducing heat input requirements and improving process efficiency, while also recirculating unused reactant back to the start of the process improving conversion efficiency. The preferred system facilitates low-energy, cost-efficient production of synthetic, or synthesized, i.e. manufactured, fuels, especially liquid e-fuels.

The invention is not limited to the embodiment(s) described herein but can be amended or modified without departing from the scope of the present invention.

1. A system for producing synthetic fuel, the system comprising a reactor,
the reactor comprising:
a fluid circuit;
means for driving fluid around said fluid circuit;
a first reaction zone for implementing a first reaction in which carbon dioxide and hydrogen react to produce carbon monoxide and water;
at least one other reaction zone for implementing a second reaction in which carbon monoxide and hydrogen react to produce a fuel precursor, and a third reaction involving synthesizing fuel from said fuel precursor;
means for introducing hydrogen into said fluid circuit;
means for introducing carbon dioxide into said fluid circuit; and
means for heating fluid in said fluid circuit,
wherein said reaction zones are inter-connected in series by said fluid circuit, and wherein fluid circuit is configured to recirculate fluid around said fluid circuit.

2. The system of claim 1, wherein said at least one other reaction zone comprises a second reaction zone for implementing said second reaction, and a third reaction zone for implementing said third reaction, or wherein said at least one other reaction zone comprises a combined reaction zone for implementing both of said second and third reactions.

3. The system of claim 1, comprising at least one reservoir for storing gas, said at least one reservoir being connected to said fluid circuit for delivery of gas into said fluid circuit and also for receiving gas from said fluid circuit, wherein said fluid circuit is preferably configured to recirculate fluid from said at least one reservoir, through said reaction zones, and back to said at least one reservoir.

4. The system of claim 1, wherein said means for introducing hydrogen into said fluid circuit comprises at least one of: means for introducing hydrogen into said fluid circuit at a location upstream of said first reaction zone, and/or means for introducing hydrogen into said fluid circuit at a location between said first reaction zone and said at least one other reaction zone.

5. The system of claim 1, wherein said means for introducing carbon dioxide into said fluid circuit comprises means for introducing carbon dioxide into said fluid circuit at a location upstream of said first reaction zone.

6. The system of claim 1, wherein said reactor further includes at least one heat exchanger arranged to transfer heat from fluid exiting one or more of said reaction zones to fluid being delivered by said fluid circuit to at least one of: at least one of said reaction zones, at least said first reaction zone, or only said first reaction zone.

7. The system of claim 6, wherein said at least one heat exchanger is arranged to transfer heat from fluid exiting said first reaction zone and/or said at least one other reaction zone to fluid being delivered by said fluid circuit to said first reaction zone.

8. The system of claim 6, wherein said at least one other reaction zone comprises a second reaction zone for implementing said second reaction, and a third reaction zone for implementing said third reaction, or wherein said at least one other reaction zone comprises a combined reaction zone for implementing both of said second and third reactions, and wherein said at least one heat exchanger comprises a first heat exchanger arranged to transfer heat from said first reaction zone to fluid being delivered by said fluid circuit to said first reaction zone, and/or a second heat exchanger arranged to transfer heat from said third reaction zone or said combined reaction zone to fluid being delivered by said fluid circuit to said first reaction zone.

9. The system of claim 6, wherein said at least one heat exchanger comprises said first heat exchanger and said second heat exchanger, and wherein said fluid circuit is configured to deliver fluid to said first reaction zone via said first and second heat exchangers.

10. The system of claim 1, wherein said heating means comprises either one or both of: a first heating apparatus located upstream of said first reaction zone and being operable to heat fluid being delivered to said first reaction zone by said fluid circuit; and a second heating apparatus located between said first reaction zone and said at least one other reaction zone, and being operable heat fluid being delivered to said at least one other reaction zone by said fluid circuit, and wherein, heating apparatus comprises at least one of: a furnace, an electric furnace, a high thermal inertia electric furnace, or an electrically powered heating apparatus.

11-12. (canceled)

13. The system of claim 1, further including means for controlling an amount of hydrogen and/or a ratio of carbon to hydrogen present in the respective reaction zone during implementation of said third reaction, in order to determine the type of fuel synthesized from said fuel precursor.

14. The system of claim 1, wherein a plurality of control zones are included in said fluid circuit at a respective different location, each control zone including at least one device for controlling at least one parameter of said fluid in accordance with control information and/or at least one parameter measurement device, the system further including a control system for controlling operation of the reactor, the control system being in communication with said control zones to provide each control zone with said control information and/or to receive parameter measurement information from the control zone, wherein, said at least one parameter comprises a respective parameter indicating any one or more of: fluid composition; fluid temperature; fluid flow rate; fluid pressure; fluid level, and wherein, said control system is configured to calculate said control information by mathematically modelling said reactor using Model Predictive Control (MPC), and/or wherein said control system is configured to determine said control information using a mathematical model of the reactor, and wherein said mathematical model comprises a neural network model whereby said control system is configured to calculate said control information using an artificial neural network.

15. (canceled)

16. The system of claim 14, wherein said plurality of control zones includes a first control zone configured to control said at least one parameter of the fluid in said first reaction zone in order to implement said first reaction.

17. The system of claim 14, wherein said at least one other reaction zone comprises a second reaction zone for implementing said second reaction, and a third reaction zone for implementing said third reaction, or wherein said at least one other reaction zone comprises a combined reaction zone for implementing both of said second and third reactions, and wherein said plurality of control zones includes a control zone configured to control said at least one parameter of the fluid in said combined reaction zone in order to implement said second reaction and said third reaction.

18. The system of claim 14, wherein said at least one other reaction zone comprises a second reaction zone for implementing said second reaction, and a third reaction zone for implementing said third reaction, or wherein said at least one other reaction zone comprises a combined reaction zone for implementing both of said second and third reactions, and wherein said plurality of control zones includes a second control zone configured to control said at least one parameter of the fluid in said second reaction zone in order to implement said second reaction.

19. The system of claim 14, wherein said at least one other reaction zone comprises a second reaction zone for implementing said second reaction, and a third reaction zone for implementing said third reaction, or wherein said at least one other reaction zone comprises a combined reaction zone for implementing both of said second and third reactions, and wherein said plurality of control zones includes a third control zone configured to control said at least one parameter of the fluid in said third reaction zone in order to implement said third reaction, or to control said at least one parameter of the fluid in said combined reaction zone in order to implement said second and third reactions.

20. The system of claim 1, further including either one or both of: means for separating the synthesized fuel from other fluid in said fluid circuit and means for introducing a carrier gas into said fluid circuit.

21. (canceled)

22. A method of producing synthetic fuel in a reactor comprising a fluid circuit, the method comprising:
introducing hydrogen into said fluid circuit;
introducing carbon dioxide into said fluid circuit;
implementing, in a first reaction zone of the reactor, a first reaction in which carbon dioxide and hydrogen react to produce carbon monoxide and water;
implementing, in at least one other reaction zone of the reactor, a second reaction in which carbon monoxide and hydrogen react to produce a fuel precursor, and a third reaction involving synthesizing fuel from said fuel precursor,
wherein said reaction zones are connected in series by said fluid circuit and wherein said method further includes recirculating fluid around said fluid circuit.

23. The method of claim 22, wherein said implementing in at least one other reaction zone comprises: implementing, in a second reaction zone of the reactor, said second reaction; and implementing, in a third reaction zone of the reactor, said third reaction; or implementing, in a combined reaction zone of the reaction, said second reaction and said third reaction.

24. The method of claim 22, further comprising controlling an amount of hydrogen and/or a ratio of carbon to hydrogen present in the respective reaction zone during implementation of said third reaction, in order to determine the type of fuel synthesized from said fuel precursor.

25. The method of claim 22, further including obtaining said hydrogen by at least one of: implementing a thermochemical cycle in which water is split into hydrogen and oxygen or sequestering said carbon dioxide from the atmosphere.
